(19) United States
(12) Patent Application Publication
(10) Pub. No.: US 2020/0352769 A1
(43) Pub. Date: Nov. 12, 2020

(54) ADJUSTING DEVICE, AND ORTHOSIS HAVING AN ADJUSTING DEVICE

(71) Applicant: Ottobock SE & Co. KGaA, Duderstadt (DE)

(72) Inventors: Matthias SCHILLING, Weißenborn-Lüderode (DE); André MÜLLER, Duderstadt (DE)

(73) Assignee: Ottobock SE & Co. KGaA, Duderstadt (DE)

(21) Appl. No.: 15/733,150

(22) PCT Filed: Nov. 30, 2018
(86) PCT No.: PCT/EP2018/083163

§ 371 Date: May 28, 2020

(30) Foreign Application Priority Data
Dec. 1, 2017 (DE) 10 2017 128 613.6

(51) Int. Cl. A61F 5/01

(52) U.S. Cl. CPC A61F 5/0102; A61F 5/0125; A61F 2005/0167; A61F 2005/0158; A61F 5/0127

(57) ABSTRACT

An adjusting device for adjustably securing at least two orthotic components to each other. The adjusting device includes a first holder which has a first fastening element for securing a first orthotic component, and a second holder which has a second fastening element for securing to a second orthotic component. The holders are mounted such that they can be moved in relation to each other and secured to each other in at least two orientations.

The invention relates to an adjusting device for adjustably securing at least two orthotic components to one another, having a first holder which has a first fastening device for securing a first orthotic component, and a second holder which has a second fastening device for fastening a second orthotic component, and to an orthosis having such an adjusting device.

Orthoses are auxiliary devices which are worn on the body of a patient. Said orthoses serve for stabilizing joints, for de-stressing a limb, for restricting a mutual relative movement between two limbs, for example by limiting extension and flexion, or else for guiding a movement. There are likewise orthoses which carry out a corrective function, for example so as to mutually align joint components or de-stress individual regions of joints in the case of osteoarthritic symptoms. Orthoses can be fastened to a limb so as to encompass joints, or else be worn on the torso of a user. Orthoses in many instances have a plurality of components which have to be mutually aligned; it is likewise necessary for the orthosis to be adapted to the user, and therefore the individual components have to be mutually oriented and aligned in order for the respective orthosis to be able to perform the predefined or desired function thereof in the best possible manner,

Standard components which are adapted to the respective function and to the respective user are often used in the manufacturing of orthoses. An important aspect in guaranteeing optimal functionality of an orthosis is the correct mutual alignment of the components and the correct fit of the orthosis on the respective user in order for the desired functions to be provided, on the one hand, and the comfort of wearing not to be restricted, on the other hand.

For adapting a foot lifting orthosis, for example, it is provided that a lower leg brace is adapted to the shape of the lower leg in that the lower leg brace is plastically shaped. A footplate is connected in an articulated manner to the lower leg brace, wherein the ankle joint is situated so as to be level with the natural ankle joint. The lower leg brace is placed on the lower leg of the patient by way of fastening straps, the foot is set on the foot shell or the foot plate and is supported by way of a spring during the swing phase. Such adapting is complex and requires vast amount of experience by an orthopedic technician when plastically deforming the solid metal braces.

U.S. Pat. No. 6,540,703 B1 relates to an orthosis for post-operative application in order to prevent luxation of the hip by way of a pelvic belt and a thigh cuff which are pivotably connected to one another by way of a joint. The joint is configured between two metal braces which are mounted firstly on the pelvic belt and then on the thigh cuff. Slots along which the pelvic belt and the thigh cuff, respectively, are mounted so as to be longitudinally displaceable are disposed in the respective braces. Semi-circular receptacles having transverse slots so as to enable rotary setting about the longitudinal extent of the respective brace are disposed on the pelvic belt and the thigh cuff.

It is an object of the present invention to provide an adjusting device and an orthosis by way of which improved mutual adapting of orthotic components can be performed so as to be able to better align an orthosis on a patient.

This object is achieved by an adjusting device having the features of the main claim and by an orthosis having the features of the further independent claim. Advantageous design embodiments and refinements of the invention are disclosed in the dependent claims, the description, and the figures.

The adjusting device for mutually adjusting at least two orthotic components, having a first holder which has a first fastening device for securing a first orthotic component, and a second holder which has a second fastening device for securing to a second orthotic component provides that the two holders are mounted so as to be able to be mutually displaced and mutually fixed in at least two orientations. On account of the design embodiment of the adjusting device from two holders which are in each case to be mutually aligned and fastenable to the fixable orthotic components it is possible to enable a mutual adjustability of the orthotic components in a plurality of planes, as a result of which the freedom of design in terms of the dimensioning and of the adjusting directions is increased. By way of the adjusting device it is possible for standardized orthotic components among themselves, or else individually adapted orthotic components such as receptacle shells of limbs and standard components, to be coupled in a very variable manner so that the functionality of the finished orthosis is guaranteed. The scope of adjustment and the adjusting direction are not limited by the constructive parameters of the orthotic components per se, but pivoting paths or displacement paths as well as the respective directions can be expanded on account of the design embodiment of the adjusting device having two holders which can in each case be secured and fixed to an orthotic component. The components of the orthoses, on account of the mutually displaceable and mutually fixable mounting in at least two different orientations in the holder, can be adapted in a simple, rapid, and reversible manner so as to be optimized for the respective patient.

One design embodiment of the invention provides that each holder has a main support on which at least one coupling portion is in each case disposed or configured, wherein the respective coupling portion protrudes from the main support. The respective orthotic component can be secured on the main support; depth setting or depth compensation can be effected by way of the coupling portions which protrude from the respective main support, optionally in combination with a longitudinal displacement capability such that the mutual spacing of the orthotic components can be varied in a simple manner via the coupling portion. Fastening devices by way of which respective orthotic components can be secured or fixed can be disposed on the respective main support. When the coupling portion protrudes orthogonally from the main support a purely orthogonal repositioning of the main supports away from one another or toward one another can be carried out in the case of a longitudinally displaceable mounting of the coupling portions. In the case of an inclined orientation of the coupling portions relative to the main support, deviating from a right angle, in particular at an obtuse angle which is larger than 90°, a superimposed movement results in the displacement along the coupling portions, in which superimposed movement the main supports are not only repositioned away from one another or toward one another, but the mutual alignment of said main supports and the spacing of the coupling portions are also adapted.

In one refinement of the invention it is provided that two mutually opposite coupling portions are in each case configured on or fastened to the main support such that a type of yoke or a U-shaped design embodiment is created, on account of which assigning the two holders to one another and thus to one another on the orthotic components is enabled in a particularly stable manner. By way of the two coupling portions that protrude from the main support a uniformly distributed transmission of force can take place by way of two mutually opposite pairs of coupling portions when securing the coupling portions, this enabling a stable mutual assignment of the orthotic components.

In a design embodiment having two mutually opposite coupling portions on a main support it is provided in one refinement that the coupling portions of a first holder have mutually facing internal faces having a clearance therebetween, wherein the coupling portions of the second holder have external sides facing away from one another. The mutual spacing of the external sides of the second coupling portions herein corresponds to the clearance between the two internal sides or internal faces of the coupling portions of the first holder. On account thereof, the second holder can be positioned between the two coupling portions of the first holder such that the first holder encompasses the second holder from the outside. It is prevented on account thereof, for example, that the second holder can move away from the first holder in an uncontrolled manner; moreover, guiding for the second holder is provided by way of the coupling portions of the first holder. The clearance between the internal faces need only substantially correspond to the external spacing of the external sides of the second coupling portions.

In one refinement of the invention it is provided that the two coupling portions are mounted on a main support so as to be able to be mutually displaced and fixed in terms of their mutual spacing on the main support, on account of which the mutual spacing of the coupling portions on the main support can be set. This facilitates the disposal of the second holder within the first holder, for example, and enables the combination of a plurality of different construction modes or dimensions of a holder and those of another holder. The two displaceable coupling portions can be preloaded in the direction toward one another, for example by way of a spring, a magnet, or a corresponding combination of a ferromagnetic part and a magnet, for example as a guide of the ferromagnetic part. On account thereof, the two coupling portions have the tendency of moving toward one another such that, for example in the case of a design embodiment of the adjusting device having an internal holder having two coupling portions and an external holder in which the coupling portions bear on the external side of the coupling portions of the first holder, a slight retaining force is exerted such that preliminary fixing of the holders to one another can take place. In principle, it is also possible for the coupling portions to be spring-loaded and to be impinged with a force in the opposite direction, thus that said coupling portions are pushed away from one another so as to push from the inside against two coupling elements of a second holder. However, this requires a guide and a detent in order for the movement to be limited should the external coupling portions fail.

The holder can be configured as an angle or a yoke, wherein the yoke-shaped design embodiment is implemented in the case of two mutually opposite coupling portions. Alternatively, the holder is configured as an angle wherein increased bending moments arise on the holder having the main support and the coupling portion.

Slots or recesses can be configured in the coupling portions and/or guides can be disposed on the coupling portions, the respective other coupling portion being mounted so as to be displaceable in said slots or recesses or on said guides. The respective coupling portion is guided in said slots or guides, wherein the slots or guides can be configured so as to be rectilinear or curved so as to enable complex movements on account of a relative mutual repositioning of the coupling portions. The recesses, slots and/or guides are preferably oriented in the longitudinal extent or transverse extent of the coupling portions so as to enable a movement of the main supports away from one another, on the one hand, and a relative mutual displacement or else a relative mutual rotation, on the other hand. In one variant of the invention it is provided that the recesses, slots and/or guides of each holder are aligned in only one orientation, wherein the recesses, slots or guides on different coupling portions are mutually oriented in a different manner, preferably so as to be mutually perpendicular, such that each coupling portion or each pair of coupling portions is responsible or provided for only one adjustment direction. This facilitates the application for the orthopedic technician or the user of the orthosis, since it is unequivocally established which repositioning is possible by way of which guide such that only one fastening device or only one fixing device for the respective coupling portion has to be released in order, for example, for a spacing of a first orthotic component, such as a brace, from the fastening element, for example a receptacle shell for a limb, to be set.

In one refinement of the invention it is provided that the holders are mounted on one another so as to be mutually rotatable so as to enable, apart from purely translatory adjustments, also a rotary mutual adjustment of the holders and thus also of the orthotic components. On account of the mutually rotatable mounting of the holders which in the respectively rotated position can also be mutually fixed, a further mutual degree of freedom of the orthotic components can be set. The coupling portions preferably have curvatures which are configured so as to be mutually corresponding such that a rotary guide is provided on account of the curvatures, for example a curvature on the external side of the internal coupling portions conjointly with a corresponding internal curvature of the internal sides of the external coupling portions. To this end, the coupling portions can be configured as lateral sub-surfaces, or on the surfaces which in each case bear on one another or are oriented toward one another have corresponding curvatures of lateral sub-surfaces, such that the main supports are mounted so as to be mutually rotatable about a common axis.

The fastening devices by way of which the orthotic components can be secured on the respective holders, in particular main supports, can be configured as form-fit elements or as force-fit elements. A thread, a screw, a clip connection, a bayonet closure, or a similar latch-fit or screw-fit device can be provided as the form-fit element; likewise a form-fitting fastening of the orthotic component to the respective holder or main support can take place by way of a plug-fit connection with a bolt. Apart from the securing by a form-fit, the orthotic component can be secured in a force-fitting manner on the holder, in particular can be clamped thereon, for example by way of a clasp or a grub screw. The fastening device can be mounted so as to be displaceable in recesses, slots and/or guides of the main support, in particular along the longitudinal extent of the main support, so as to enable a further dimension in terms of adjustment. The orthotic component per se can likewise be mounted so as to be adjustable within the respective fastening device, for example can be displaceably mounted so as to enable a stepless adjustment in the case of a mounting by clamping. Fixing at discrete spacings at predetermined locations is preferably achieved by way of form-fitting locking and fastening of the orthotic components on and to the fastening device, respectively.

One of the components can be configured as an orthotic brace, while another component can preferably be configured as a support shell or receptacle or a fastening device for a limb or the torso. The component receiving the limb or the torso can be fixed to the limb by way of fastening elements such as tensioning straps, hook and loop fasteners, or the like. The orthotic brace can be coupled in an articulated manner to a further orthotic component, for example a further orthotic brace, which can likewise be mutually coupled by way of the adjusting device and a support shell. For example, a leg orthosis can be secured on the thigh and on the lower leg by way of respective support shells. The support shells are fastened at the position which is in each case the most favorable for the user. The coupling to a standardized leg orthosis having a thigh brace, an orthotic knee joint, and a lower leg brace is then established by way of the adjusting device. The positioning of the orthotic joint relative to the natural knee joint takes place in an optimized position which by way of the adjusting device having the multiplicity of setting possibilities can readily take place both in the longitudinal extent of the respective orthotic brace as well as in terms of the spacing, the rotation of the receptacle shell relative to the longitudinal extent of the respective orthotic brace, as well as the height adjustment and the adjustability of the spacing of the orthotic brace relative to the thigh and the lower leg.

One variant of the invention provides that alignment markings or graduations are disposed or configured on the coupling portions and/or the main support so as to provide the user or the orthopedic technician with indications pertaining to a predefined adjustment, on the one hand, and to enable a setting once found to be easily retrieved, for example after the replacement of a component, on the other hand. Different alignments can also be tested for experimental purposes, wherein said alignment settings can be readily identified and recorded by means of the markings such that quantitative and qualitative statements pertaining to the respective mutual orientation of the components can be made.

Locking elements which mutually fix the respective holders are disposed or configured on the holders. On account thereof, releasable mutual fixing of the holders and thus of the orthotic components is achieved. The coupling portions of the holders can be mutually fixed by way of the locking elements so that an alignment of the orthosis with the limbs or the torso can readily take place. The locking elements can be configured as screws, a thread with a nut, clamping elements, clamping toggles and/or wedges, wherein the locking elements can be guided in guides, slots, or recesses of the coupling elements so as to achieve a compact construction.

Exemplary embodiments of the invention will be explained in more detail by means of the appended figures. The same reference signs refer to identical components. In the figures:

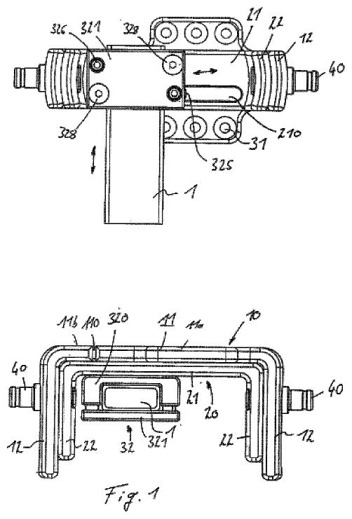

FIG. 1—shows a lateral view and a plan view of an adjusting device in an initial position;

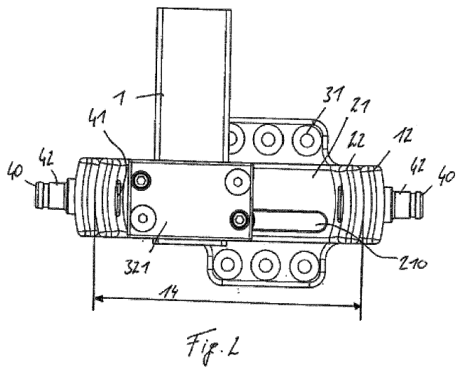

FIG. 2—shows a lateral view according to FIG. 1, having an adjusting device which is repositioned along an orthotic brace;

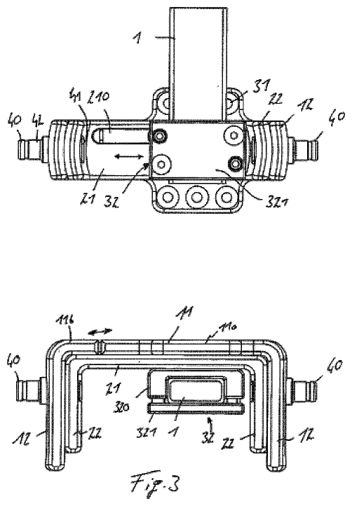

FIG. 3—shows a lateral view and a plan view of an orthotic component displaced along a main support;

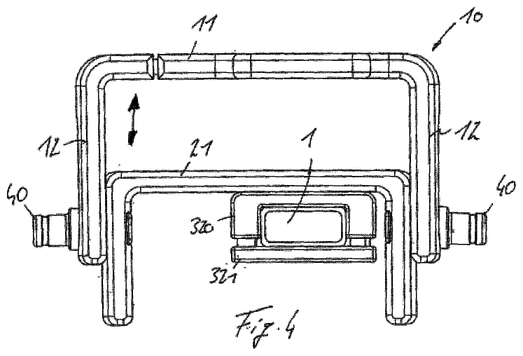

FIG. 4—shows a lateral view and a plan view of an adjusting device according to FIG. 3 which has been adjusted in terms of depth;

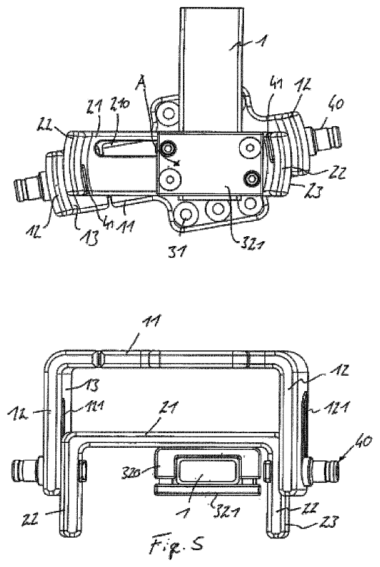

FIG. 5—shows a lateral view and a plan view of an adjusting device which has been adjusted in terms of angle;

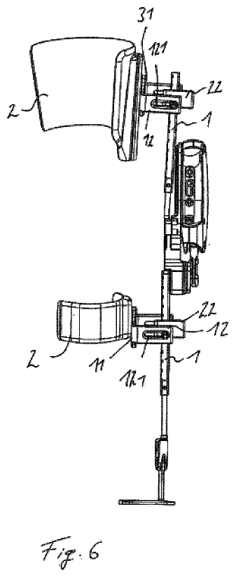

FIG. 6—shows a frontal view of a leg orthosis;

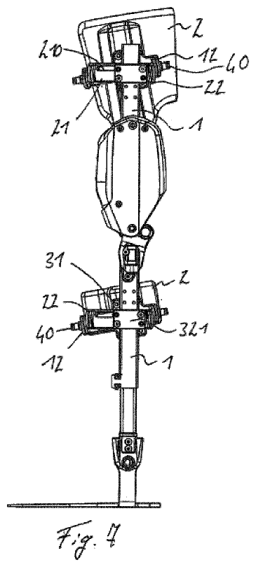

FIG. 7—shows a lateral view of an orthosis according to FIG. 6;

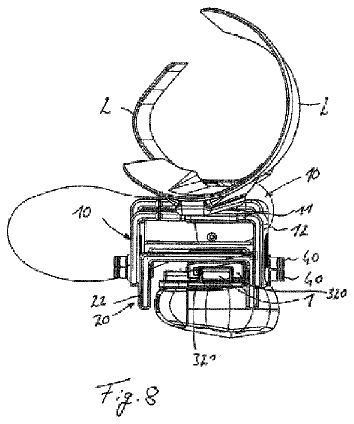

FIG. 8—shows a plan view of an orthosis according to FIG. 6;

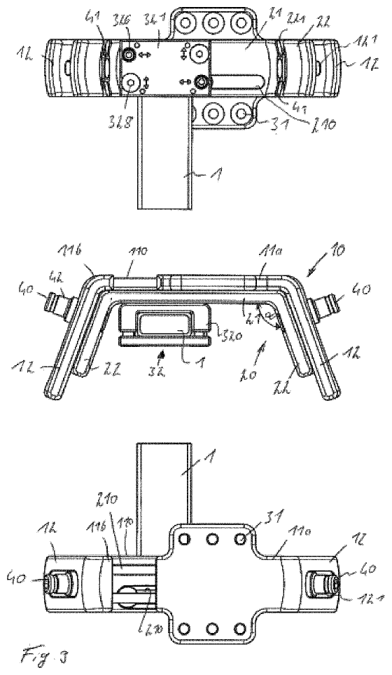

FIG. 9—shows three illustrations of a first variant of an adjusting device;

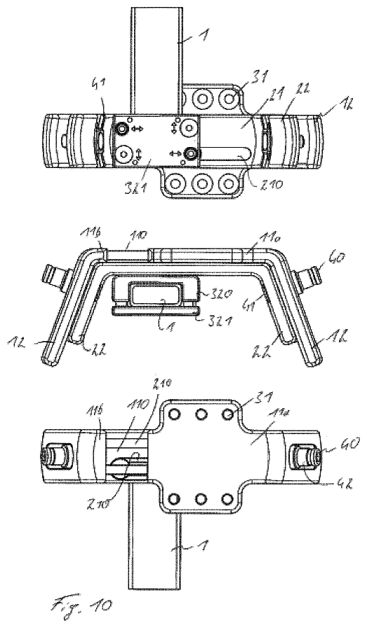

FIG. 10—shows three illustrations of a variant of FIG. 9 having a displaced first component;

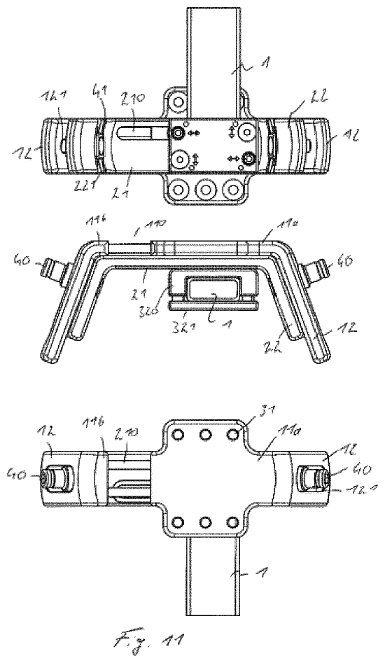

FIG. 11—shows a variant of FIG. 9 having a displaced fastening device;

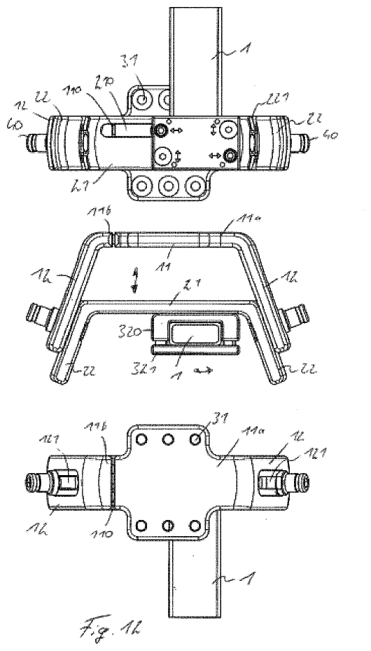

FIG. 12—shows a variant of FIG. 11 having displaced holders; and

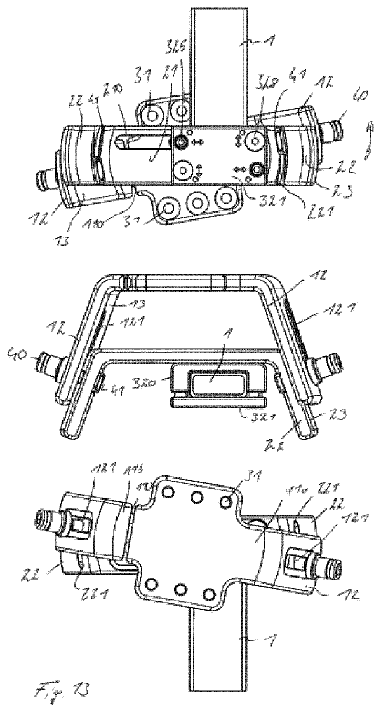

FIG. 13—shows a variant of FIG. 12 having mutually rotated holders.

FIG. 1 shows two views of an adjusting device for adjustably securing two orthotic components of which only a first orthotic component 1 is shown in the form of an orthotic brace. The adjusting device has two holders 10, 20 and in FIG. 1 is shown in a lateral view in the upper illustration and shown in a plan view in the lower illustration. The first holder 10 is constructed in two parts and has a main support 11 which is configured so as to be substantially rectilinear and from which two coupling portions 12 protrude orthogonally. The main support 11 of the first holder 10 is configured in two parts and has a first part 11a in which guides 110, for example pins, of the second part 11b are received. The second part 11b is guided along said guides 110 so as to be longitudinally displaceable relative to the first part 11a such that the spacing between the two coupling portions 12 of the first holder 10 can be set. The two parts 11a, 11b can be preloaded toward one another by way of magnets, springs, or similar devices (not illustrated) such that the two parts 11a, 11b are at all times moved back to an initial position and are loaded with a force in the direction toward the initial position. The first holder 10 is configured so as to be U-shaped in plan view and on the main support 11 has fastening devices 11 in the forms of bores for screw receptacles. The fastening devices 31 serve for securing a second orthotic component on the first holder 10.

It can be seen in the lower illustration of FIG. 1 that the second holder 20 is constructed so as to correspond substantially to the first holder 10 and has a main support 21 as well as two coupling portions 22 which protrude orthogonally from said main support 21. The second support 20 is embodied in one part and disposed within the first holder 10 between the coupling portions 12 thereof. The two main supports 11, 21 bear on one another; the respective coupling portions 12, 22 being aligned in the same direction. The second holder 20 is also configured so as to be U-shaped and is likewise produced from a metallic material. The two coupling portions 22 protrude orthogonally from the main support 21. A fastening device 32 for the orthotic brace 1 is disposed on the main support 21 between the coupling elements 22. The fastening device 32 is mounted so as to be longitudinally displaceable within two recesses 210 along the longitudinal extent of the main support 21, only one recess 210 of said two recesses 210 being visible. The second recess 210 is obscured by the second fastening device 32. Two guide pins 210 are mounted in the respective slot-shaped guides 210 and can be fixed in a clamping manner in the guides 210 by way of hex socket screws 326.

The brace 1 per se is held in a force-fitting manner between a U-shaped holder 320 and a clamping plate 321 by way of two screws 328, and within the second fastening device 32 can be displaced in a stepless manner along the longitudinal extent of the brace 1. The displacement capability is indicated by the double arrow in the upper illustration of FIG. 1. On account of the clamping fastening, the position of the adjusting device having the two holders 10, 20 can be set in a stepless manner along the longitudinal extent of the brace 1.

It can be seen in the upper illustration of FIG. 1 that the coupling portions 12, 22 have a curved shape. The coupling portions 12, 22 are shaped as lateral sub-surfaces such that the two holders 10, 20 can be pivoted in a mutually relative manner about a central pivot axis. To this end, guides in the form of recesses are disposed within the second holder 20 in the exemplary embodiment illustrated, the longitudinal extent of said recesses running orthogonally to the longitudinal extent of the coupling portions 22. This functional mode will be explained in more detail later. Locking elements 40 in the form of threaded bolts are disposed within the guides, mutual securing of the holders 10, 20 in a clamping manner being able to take place by way of said locking elements 40.

FIG. 2 shows the locking device according to FIG. 1 in a height-adjusted state in which the brace 1 has been displaced upward within the fastening device 32. The adjusting device is thus repositioned downward relative to the first orthotic component 1 and is fixed therein in a clamping manner by way of the screws 328. It can be seen in FIG. 2 that a clearance 14 is configured between the outer coupling portions 12, the second holder 20 by way of the coupling portions 22 being disposed within said clearance 14. The curved external surfaces or external sides 23 of the coupling portions 22 of the second holder 10 bear on the correspondingly curved internal surfaces or internal sides 13 of the coupling portions 12 of the first holder, specifically such that the holders 10, 20 in a state not fixed by a locking element 40 can slide into one another and along one another.

The locking elements 40 have a bolt with a head 41 and a nut 42, by way of which the coupling portions 12, 22 of the holders 10, 20 can be mutually fixed in a clamping manner.

The adjusting device in FIG. 3 is illustrated in a lateral view in the upper illustration and in a plan view in the lower illustration. The second fastening device 32 is displaced from the position according to FIG. 2 to the right along the guides 210. It can be seen in FIG. 3 that a second guide 210 is guided in the main support 21 of the second holder 20. The two guides 210 are disposed on different levels and so as to be mutually offset in the main support 21 such that a displacement can take place across the entire length of the main support 21. The fastening device 32 can thus be displaced across the entire internal spacing of the coupling portions 22 between the two coupling portions 22 of the second holder 20. The double arrow on the first holder 10 indicates the displacement capability of the second part 11b. The locking elements 40 can be permanently secured on the second holder 20 by way of the nut 42 and the bolt with the screw head 41.

FIG. 4 shows a further variant of the invention in which longitudinal guides 121 are incorporated within the first coupling portions 12 of the first holder 10 such that the second holder 20 can be displaced along the longitudinal extent of the coupling portions 12. The displacement capability is indicated by the double arrow.

The spacing between the main supports 11, 12 can be varied and set on account of the mounting of the second holder 20 in the longitudinal guides along the coupling portions 12 in the first holder 10. It is possible for the desired mutual position of the holders 10, 20, and thus also of the two orthotic components 1, 2 in each case to be set by tightening the locking elements 40.

FIG. 5 in a lateral view in the upper illustration and in a plan view in the lower illustration shows the adjusting device in a position in which the holders 10, 20 are mutually rotated. Guides which cannot be seen and in which the screw heads 41 slide along are incorporated within the coupling portions 22 of the second holder 20 so as to be perpendicular to the longitudinal extent of said coupling portions 22. The screw heads 41 are guided in grooves within the coupling portions 22 such that said screw heads 41 do not project beyond the internal surfaces of the coupling portions 22. It can be seen in the upper illustration of FIG. 5 that the two holders 10, 20 are mutually pivoted about a pivot axis A. The surfaces 23, 13 of the respective coupling portions 22, 12 that are provided with an identical radius and slide on one another bear on one another; the main supports 11, 21 are mutually tilted on account of which the first holder 10 can be pivoted relative to the second support 20 and thus also relative to the orthotic brace 1 which is assembled on the second holder 20. The longitudinal guides 121 within the first coupling portions 12 of the first holder 10 can be seen in the lower illustration of FIG. 5, the locking elements 40 being able to be guided along said longitudinal guides 121. The guides 121 are configured as slots and have encircling depressions within which the nuts 42 are guided along.

The adjusting device according to FIGS. 1 to 5 by way of the longitudinal guides 121 in the first coupling portions 12 thus enables a mutual adjustment of the two holders 10, 20 along the longitudinal extent of the respective coupling portions 11, 21, and by way of the guides within the coupling portions 22 of the second holder 20 rotation about the pivot axis A, said guides within the coupling portions 22 running so as to be orthogonal to the longitudinal extent of the first guides 121 and orthogonal to the longitudinal extent of the coupling portions 22. Moreover, a displacement capability of the first component 1 within the two coupling portions 22 of the second holder 20 along the guides 210 is possible by way of the fastening device 32. Additionally, the orthotic brace 1 can be displaced within the clamping mounting of the fastening device 32 so that a total of three translatory degrees of freedom and one rotary degree of freedom are covered by the adjusting device such that manifold mutual setting possibilities of the components, for example between an orthotic brace and a receptacle shell, are enabled by the adjusting device.

A leg orthosis having a thigh brace, a lower leg brace, and a foot part is illustrated in a frontal view in FIG. 6. The foot part by way of an artificial ankle joint is pivotably mounted on the lower leg brace; the lower leg brace is pivotably mounted on the thigh brace by way of an orthotic knee joint. The respective orthotic brace represents the first orthotic component 1 which has already been illustrated in FIGS. 1 to 5 and explained in the context thereof. The adjusting device having the two holders 10, 20 can be displaced along the longitudinal extent of the respective orthotic brace 1 by releasing the screws of the second fastening device 32 and subsequently fixed again in a stepless manner at the desired height. The two longitudinal guides 121 in the first holder 10, by way of which a spacing of the in each case second component 2 from the orthotic brace 1 can be set, can be seen in the frontal view. The second orthotic component 2 in the exemplary embodiment illustrated is in each case a receptacle shell for a limb, here a thigh receptacle shell and a lower leg receptacle shell. The respective receptacle shell 2 is secured on the thigh, or on the lower leg, respectively, by way of fastening devices (not illustrated) such as belts or buckles such that a coupling between the limb and the functional component of the orthosis, specifically the guide rails 1 having the joints and optionally a resistance device, the latter being electronically controlled, can be established. The in each case second component 2 is coupled to and secured on the first holder 10 by way of the first fastening device 31 in the form of screws or screw bores. It can be gathered from FIG. 6 that the holders 10, 20 are mutually tilted. The tilting is more clearly shown in FIG. 7. The first holder 20 is oriented so as to be substantially horizontal, that is to say that the main support 10 has an upper edge which runs so as to be substantially horizontal. To this end, the upper edge of the first main support 11 of the first holder 10 is tilted in a counter-clockwise manner by approx. 20°, this corresponding approximately to the maximum tilting angle about the common axis A. Two adjusting devices having in each case two holders 10, 20 are disposed in the orthosis so as to align the receptacle shells 2 relative to the orthotic braces 1 and to one another, and to enable optimized adapting to the patient.

The orthosis having the thigh brace 1, a total of four holders 10, 20, as well as the two second orthotic components 2 in the form of receptacle shells, is illustrated in a plan view in FIG. 8.

FIG. 9 shows three illustrations of a variant of the invention in a frontal view, a plan view, and a rear view. The same reference signs refer to identical components. The fundamental construction of the adjusting device corresponds to that illustrated in FIG. 1. To this extent, the explanations pertaining to the latter apply in analogous manner. As opposed to the embodiment according to FIG. 1, the holders 10, 20 are not equipped with coupling portions 12, 22 which are oriented at a right angle to a main support 11, 21, but between the main support 11, 21 and the coupling portions 12, 22 have an angle α which is larger than 90° and forms an obtuse angle. The angle α between the main support 11, 21 and the respective coupling portions 12, 22 is preferably configured in an angular range between 91° and 130°. By virtue of the design embodiment of the first holder 10 in two parts it is possible for the main support 11 of the first holder 10 to be varied in terms of the length thereof. The main support 11 of the first holder 10 is illustrated at the maximum length thereof in FIG. 9; the second holder 10 by way of the main support 21 thereof bears on the main support 11 of the first holder 10. The second fastening device 32, when viewed in the frontal view and in the plan view, is situated to the extreme left within the recesses 210 in the second holder 20. The first orthotic component 1 in the form of a brace is held in a clamping manner in the second fastening device 32. The screws 328 prevents a displacement capability of the first orthotic component 1 in the longitudinal extent thereof, toward the top or the bottom in the upper illustration of FIG. 9. This is indicated by the double arrows at the screws 328. The screws 326 secure the second fastening device 32 on the second holder 20. The second fastening device 32 is mounted so as to be displaceable to the right and the left in the recesses 210 and by releasing and tightening the screws 326 can be displaced and fixed in a clamping manner in any arbitrary position between the extreme left position, as illustrated in FIG. 9, and an extreme right illustration, as shown in FIG. 11.

The distance between the main supports 11, 21 can be realized by repositioning the second holder 20 relative to the first holder 10 along the recesses 121 in the two coupling portions 12 of the first support 10. Releasable and stepless setting along the recesses 121 can be performed by way of the locking elements 40 having the head 41 and the nut 42. The second holder 20 in the illustration according to FIG. 9 is in a position of maximum proximity to the first holder 10.

FIG. 10 shows a variant of FIG. 9, in which the first orthotic component 1 in the form of an orthotic brace is displaced within the second fastening device 32. For example, setting the height of the second orthotic component, which is not illustrated, relative to the first orthotic component 1 is possible by way of this displacement and fixing by the screws 326.

FIG. 11 shows a further variant of the invention in which the second fastening device 32 has been displaced to the extreme right, when viewed in the frontal view and the plan view. It can be seen that the left recess 210 is disposed above the first recess 210 which can be seen in FIGS. 9 and 10.

FIG. 12 shows a variant of FIG. 11, in which it can be seen, in particular in the plan view, thus in the illustration in the center of FIG. 12, that the second holder 20 is in a position of maximum remoteness with respect to the main supports 11, 21. The two parts 11a, 11b of the holder 10, by virtue of the inclined position of the coupling portions 12, 22 relative to the respective main support 11, 21 are moved toward one another in a longitudinal displacement of the second holder 20 along the recess 121 in the coupling portions 12 of the first support 10, and said two parts 11a, 11b at a maximum distance of the main supports 11, 21 are situated in a state of maximum proximity and may bear on one another. The first fastening device 31 can thus be adjusted in terms of depth relative to the second fastening device 32; the first orthotic component 1 can thus be repositioned in a second direction or orientation in relation to the second orthotic component 2. The directions of movement are indicated by the double arrows in FIG. 12. The two orthotic components 1, 2 can be mutually displaced or repositioned in different directions which are oriented so as to be mutually perpendicular.

An adjusting device which has been adjusted in angular terms similar to that in FIG. 5 is shown in FIG. 13. Recesses 221 in the form of tips which, conjointly with the radiused design embodiment of the external side 23 of the coupling portions 22 and the radiused internal side 13 of the coupling portions 12, enable a pivoting movement about an axis which is perpendicular to the sheet plane are configured in the second holder 20 in the two coupling portions 22. The potential pivoting movement is indicated by the curved double arrow in the upper illustration. When the second orthotic component 2 is firmly fastened to the first fastening device 31, repositioning in all three translatory degrees of freedom and rotating about one degree of freedom can thus be overall achieved such that the orthotic components 1, 2 can be rapidly, simply and steplessly mutually aligned and adapted to the respective patient. As an alternative to a stepless and clamping adjustment of the holders 10, 20 or of the fastening devices 31, 32 relative to one another, graduated repositioning movements having form-fitting locking mechanisms by way of latching devices can also be configured.

1. An adjusting device for adjustably securing at least two orthotic components to one another, the adjusting device comprising:
a first holder which has a first fastening device for securing a first orthotic component;
a second holder which has a second fastening device for securing to a second orthotic component, the first and second holders being mounted to be mutually displaced and mutually fixed in at least two orientations.

2. The adjusting device as claimed in claim 1, wherein each holder has a main support on which at least one coupling portion is in each case disposed or configured, wherein and the at least one coupling portion protrudes from the main support.

3. The adjusting device as claimed in claim 2, wherein the at least one coupling portion protrudes orthogonally or at an obtuse angle from the main support.

4. The adjusting device as claimed in claim 2, wherein the at least one coupling portion for each holder includes two mutually opposite coupling portions that are in each case configured on or fastened to the main support.

5. The adjusting device as claimed in claim 4, wherein the coupling portions of the first holder have mutually facing internal faces having a clearance therebetween, and the coupling portions of the second holder have external sides facing away from one another, and a spacing of the external sides of the second coupling portions of the second holder correspond to the clearance between the internal faces.

6. The adjusting device as claimed in claim 4, wherein the coupling portions for each holder are mounted on the main support so as to be able to be at least one of mutually displaced and/or fixed in terms of their mutual spacing.

7. The adjusting device as claimed in claim 6, wherein the coupling portions for each holder are preloaded in a direction toward one another.

8. The adjusting device as claimed in claim 2, wherein at least one of slots or recesses are configured in the coupling portions and guides are disposed on the coupling portions, the respective other coupling portion being mounted so as to be displaceable in the slots or recesses or on the guides.

9. The adjusting device as claimed in claim 8, wherein at least one of the recesses, slots and guides are oriented in a longitudinal extent or transverse extent of the coupling portions.

10. The adjusting device as claimed in claim 9, characterized in wherein the at least one of the recesses, slots and/or guides of each holder are aligned in only one orientation, and the at least one of the recesses, slots or guides on different coupling portions are mutually oriented in a different manner.

11. The adjusting device as claimed in claim 1, wherein the holders are mounted on one another so as to be mutually rotatable.

12. The adjusting device as claimed in claim 2, wherein the coupling portions have curvatures which are configured so as to be mutually corresponding.

13. The adjusting device as claimed in claim 12, wherein the coupling portions are configured as lateral sub-surfaces, or comprise lateral sub-surfaces, and the main supports are mounted so as to be rotatable about a common axis.

14. The adjusting device as claimed in claim 1, wherein the fastening device is configured as a form-fit element or as a force-fit element.

15. The adjusting device as claimed in claim 1, wherein the fastening device is mounted on the main support so as to be displaceable in at least one of recesses, slots and/or guides.

16. The adjusting device as claimed in claim 1, wherein one of the components is configured as an orthotic brace.

17. The adjusting device as claimed in claim 1, wherein one of the components is configured as a support shell or a receptacle for a limb or the torso.

18. The adjusting device as claimed in claim 2, further comprising alignment markings or graduations are disposed or configured on at least one of the coupling portions and on the main support.

19. The adjusting device as claimed in claim 1, further comprising locking elements which mutually fix the holders and are disposed on the holders.

20. The adjusting device as claimed in claim 19, wherein the locking elements are configured as at least one of screws, a thread with a nut, a clamping element, a toggle, and a wedge.

21. The adjusting device as claimed in claim 19, wherein the locking elements are guided in the guides, slots or recesses of the coupling elements.

22. An orthosis having at least two orthotic components and an adjusting device as claimed in claim 1.
